(19) United States
(12) Patent Application Publication
(10) Pub. No.: US 2013/0137864 A1
(43) Pub. Date: May 30, 2013

(54) PROCESS FOR THE PREPARATION OF LINEZOLID

(71) Applicants: Jayaraman Venkat Raman, Vadodara (IN); Dhiraj Rathod, Vadodara (IN); Irfan Vohra, Vadodara (IN); Mangesh Chavan, Vadodara (IN); Manoj Ahirrao, Vadodara (IN); Ravi Ponnaiah, Vadodara (IN)

(72) Inventors: Jayaraman Venkat Raman, Vadodara (IN); Dhiraj Rathod, Vadodara (IN); Irfan Vohra, Vadodara (IN); Mangesh Chavan, Vadodara (IN); Manoj Ahirrao, Vadodara (IN); Ravi Ponnaiah, Vadodara (IN)

(21) Appl. No.: 13/518,503

(22) PCT Filed: Dec. 9, 2010
(86) PCT No.: PCT/IB10/55678

§ 371 Date: Jun. 22, 2012

(30) Foreign Application Priority Data
Dec. 26, 2009 (IN) 2997/MUM/2009
Mar. 16, 2010 (IN) 692/MUM/2010

(51) Int. Cl. C07D 263/06

(52) U.S. Cl. CPC C07D 263/06
USPC 544/137

(57) ABSTRACT

The present invention provides an improved process for the preparation of Linezolid of formula (D. The present invention relates to preparation of intermediate (R)—N-[[3-[3-fluoro-4-morpholinyl]phenyl|-2-oxo-5-oxazolidinyl]methanol of formula (II), Linezolid amine of formula (Ia) and their use in the preparation of Linezolid. The present invention further provides process for the preparation of Form I of Linezolid of formula (I).

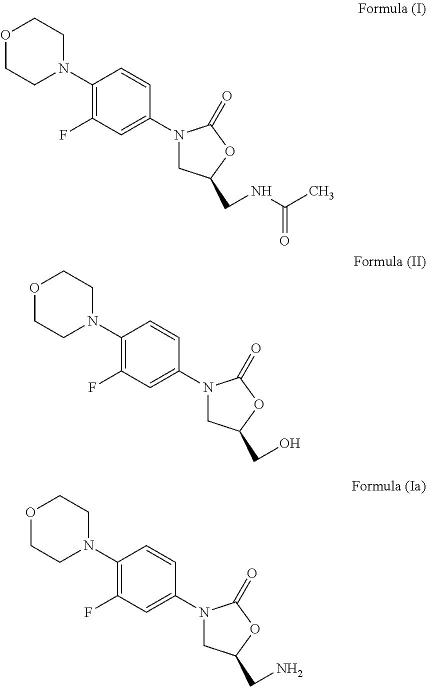 [SMILES: CC(=O)NC[C@H]1CN(c2ccc(N3CCOCC3)c(F)c2)C(=O)O1 ; O=C1O[C@@H](CO)CN1c1ccc(N2CCOCC2)c(F)c1 ; NC[C@H]1CN(c2ccc(N3CCOCC3)c(F)c2)C(=O)O1]

### FIELD OF THE INVENTION

The present invention provides an improved process for the preparation of Linezolid of formula (I).

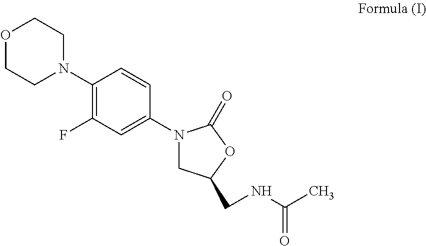 [SMILES: CC(=O)NC[C@H]1CN(c2ccc(N3CCOCC3)c(F)c2)C(=O)O1]

The present invention relates to preparation of intermediate (R)—N—[[3-[3-fluoro-4-morpholinyl]phenyl]-2-oxo-5-oxazolidinyl]methanol of formula (II), Linezolid amine of formula (Ia) and their use in the preparation of Linezolid.

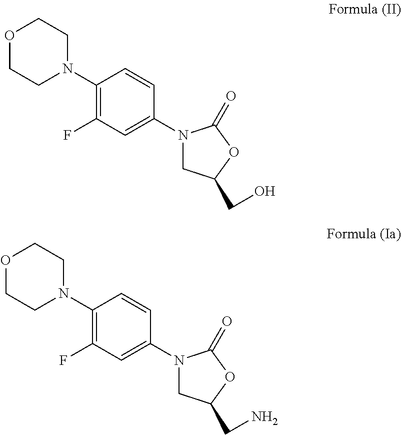 [SMILES: O=C1O[C@@H](CO)CN1c1ccc(N2CCOCC2)c(F)c1 ; NC[C@H]1CN(c2ccc(N3CCOCC3)c(F)c2)C(=O)O1]

The present invention further provides process for the preparation of Form I of Linezolid of formula (I).

### BACKGROUND OF THE INVENTION

Linezolid is chemically known as N—[[(5S)-3-[3-Fluoro-4-(4-morpholinyl)phenyl]-2-oxo-5-oxazolidinyl]methyl]acetamide and marketed by Pfizer in US under brand name Zyvox. Linezolid is a synthetic antibacterial agent of the oxazolidinone class. It is used for the treatment of infections caused by multi-resistant bacteria including streptococci and methicillin-resistant Staphylococcus aureus.

Linezolid was first disclosed in U.S. Pat. No. 5,688,792. The process for synthesis is as disclosed in Scheme-I

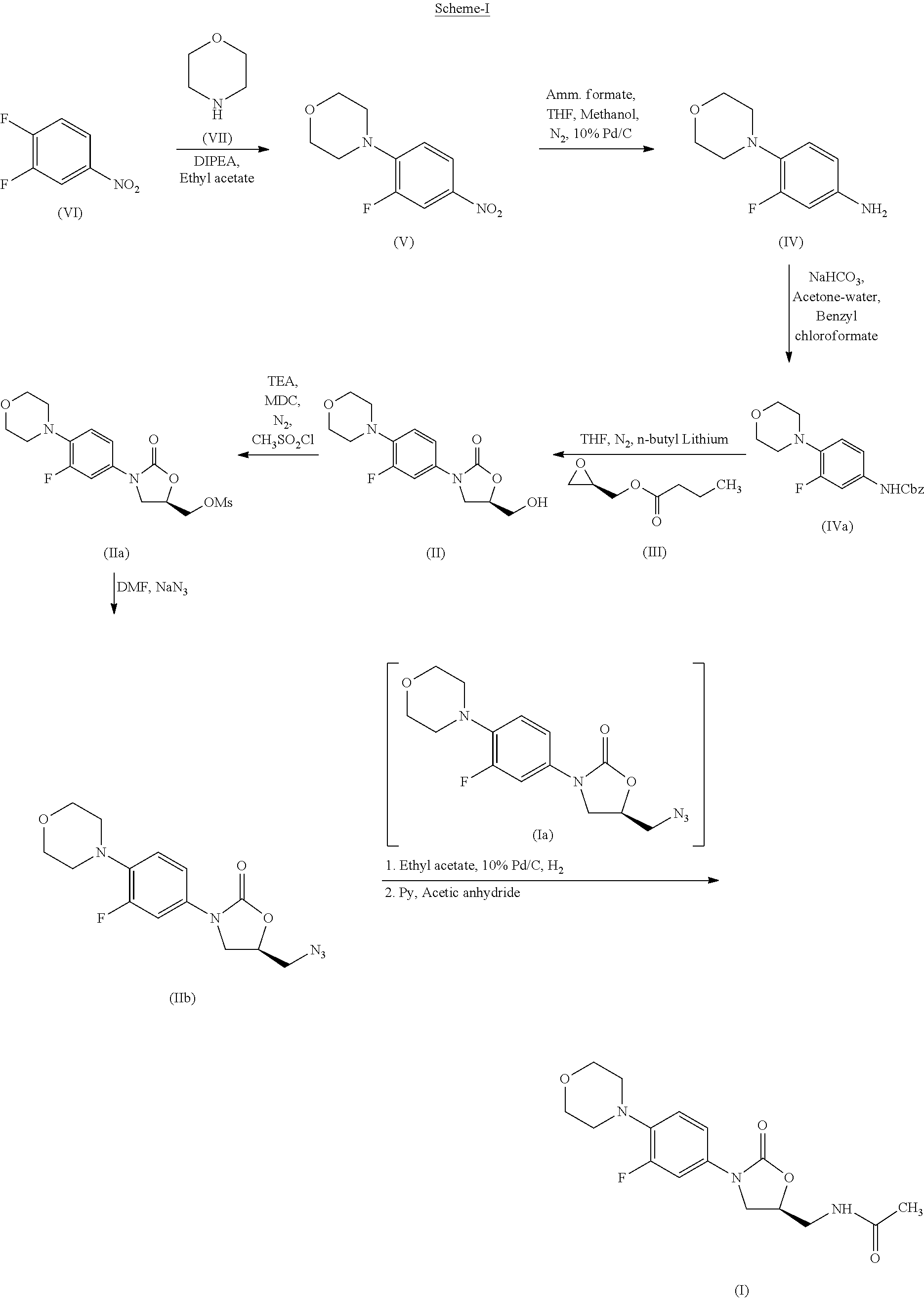

In the process disclosed above the key intermediate (II) is obtained by reacting N-carbobenzyloxy-3-fluoro-4-morpholinyl aniline of formula (IVa) with (R)-glycidyl butyrate of formula (III) in the presence of n-butyl lithium to obtain compound of formula (II). Compound of formula (II) is converted to (IIb) by tosylation and reaction with sodium azide. Reduction of Linezolid azide of formula (IIb) in the presence of palladium/carbon in ethyl acetate solvent to obtain Linezolid amine (Ia), which is further treated with acetic anhydride in presence of pyridine to obtain Linezolid of formula (I). The purification process involves chromatography and separating the desired fraction, followed by evaporation and triturating the product to obtain pure Linezolid.

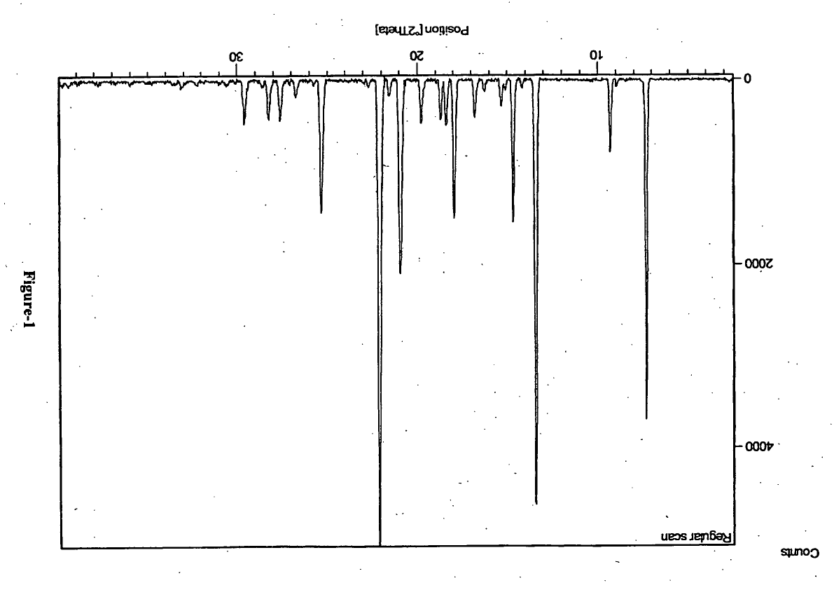

The polymorphic form obtained by following process disclosed in U.S. Pat. No. 5,688,792 is designated as Form I. FIG. 1 depicts the PXRD graph of Form I obtained by following prior art process.

Disadvantage of the process disclosed in U.S. Pat. No. 5,688,792 is that it involves use of n-butyl lithium. Due to its explosive nature it is difficult to handle at plant scale. Also, the said reaction is carried out at temperature of −78° C., which is difficult to attain during commercial production. Further the intermediate obtained requires purification by column chromatography. Column chromatography is a cumbersome technique and difficult to practice during commercial scale production. The above described process to obtain pure Linezolid results in very low yields. Further, such a process is difficult to follow at commercial level. Also, practice of chromatographic techniques requires large quantities of solvent and its subsequent recovery which increases the overall cost of production.

The process for the preparation of Linezolid is also disclosed in Journal of Medicinal Chemistry (1996), 39(3), 673-9, U.S. Pat. Nos. 6,492,555, 5,837,870, 6,887,995, 7,307,163, 7,429,661, etc.

None of the above mentioned prior arts offer simple and cost effective method for the production of compound of formula (I) from compound of formula (Ia). Nor any of the prior art process describes a process for preparation of Form I of Linezolid. Therefore, there is need to develop an efficient method, which is simple, cost-effective and commercially scalable for synthesis of Linezolid of formula (I) from Linezolid amine of formula (Ia). Further, it would be desirable to develop a process for preparation of Form I of Linezolid which is reproducible.

### OBJECT OF THE INVENTION

It is an object of the present invention to provide a process for the preparation of Linezolid formula (I).

Another object of the present invention is to provide process for the preparation of compound of formula (II).

Another object of the present invention is provided a process for preparing Form I of Linezolid of formula (I)

### SUMMARY OF THE INVENTION

An aspect of the present invention provides a process for the preparation of Linezolid of formula (I) comprising a step of

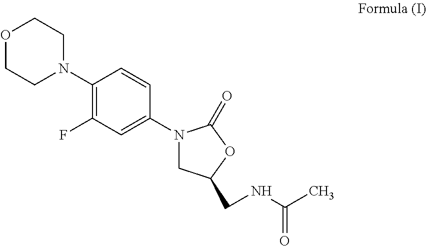 [SMILES: CC(=O)NC[C@H]1CN(c2ccc(N3CCOCC3)c(F)c2)C(=O)O1]

reacting compound of formula (IV), wherein R is Hydrogen or Nitrogen protecting group, with compound of formula (III) in presence of n-butyl lithium and n-butanol in a suitable solvent to obtain compound of formula (II)

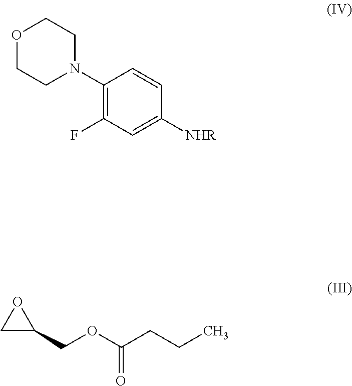

Another aspect of the present invention provides an improved process for the preparation of compound of formula (II) comprising of

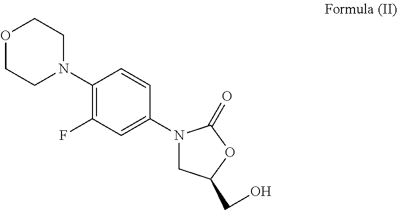 [SMILES: O=C1O[C@@H](CO)CN1c1ccc(N2CCOCC2)c(F)c1]

reacting compound of formula (IV), wherein R is Hydrogen or Nitrogen protecting group, with compound of formula (III) in presence of n-butyl lithium and n-butanol in a suitable solvent to obtain compound of formula (II)

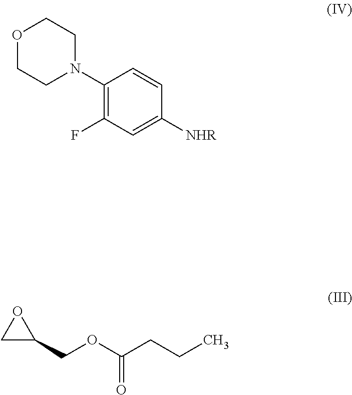

Yet another aspect of present invention provides a process for preparation of Linezolid and its key intermediate which is simple, safe, cost-effective and easy to follow at commercial scale.

In one embodiment of the present invention is provided a process for the preparation of Linezolid of formula (I) comprising,

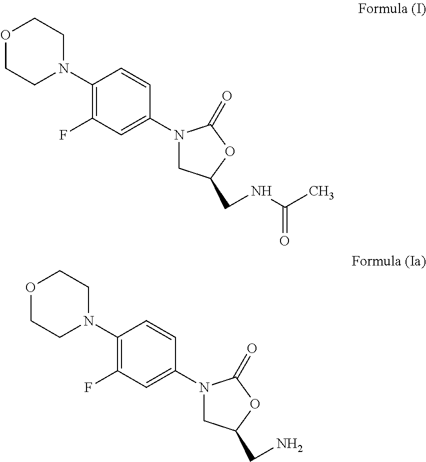 [SMILES: CC(=O)NC[C@H]1CN(c2ccc(N3CCOCC3)c(F)c2)C(=O)O1 ; NC[C@H]1CN(c2ccc(N3CCOCC3)c(F)c2)C(=O)O1]

acylating Linezolid amine of formula (Ia) using acylating agent in the presence of ketonic solvent.

In another embodiment of the present invention is provided a process for preparing Form I of Linezolid of formula (I) comprising steps of:

(a) acylating Linezolid amine of formula (Ia) using acylating agent in the presence of ketonic solvent.

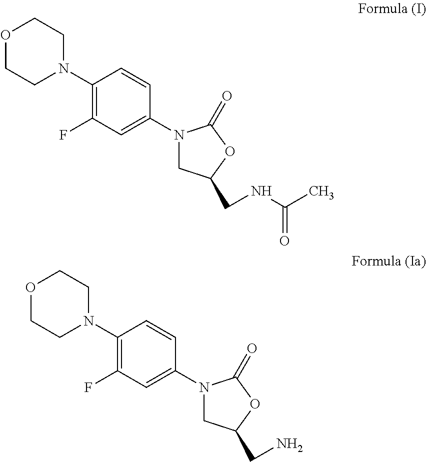 [SMILES: CC(=O)NC[C@H]1CN(c2ccc(N3CCOCC3)c(F)c2)C(=O)O1 ; NC[C@H]1CN(c2ccc(N3CCOCC3)c(F)c2)C(=O)O1]

(b) crystallizing Linezolid obtained in step (a) from suitable solvent.

Linezolid obtained by the process of present invention has content of (R)-enantiomer less than about 0.1% and bis-Linezolid content less than 0.15%. Further the purity of Linezolid is more than 99% and the yield of the reaction is high.

Therefore, the process of present invention can be employed advantageously by avoiding the cumbersome and lengthy procedure of chromatography.

### BRIEF DESCRIPTION OF THE INVENTION

FIG. 1: PXRD graph of Form I obtained by following prior art process.

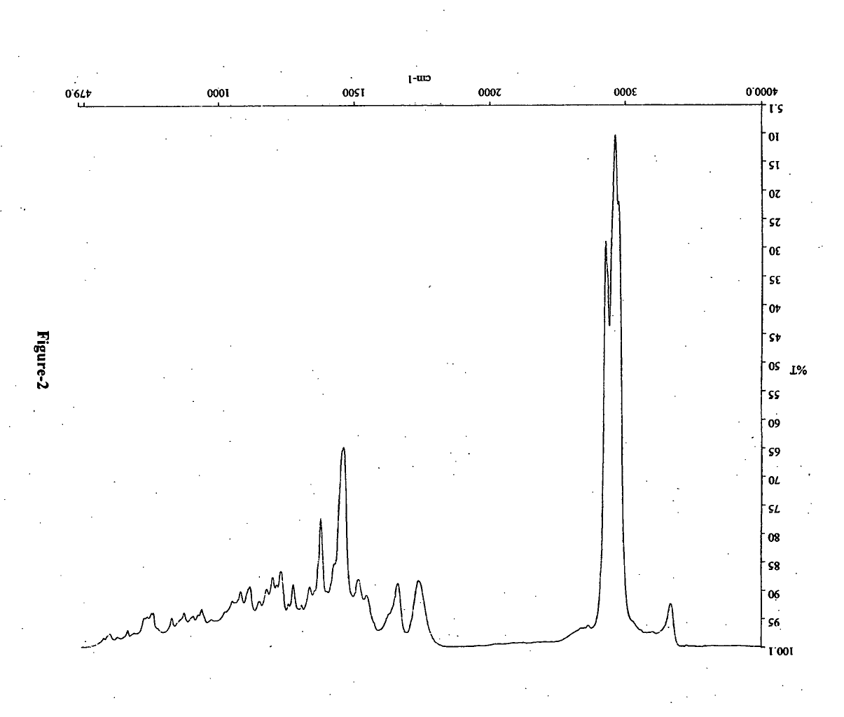

FIG. 2: FTIR (Nujol) Form I obtained by following prior art process.

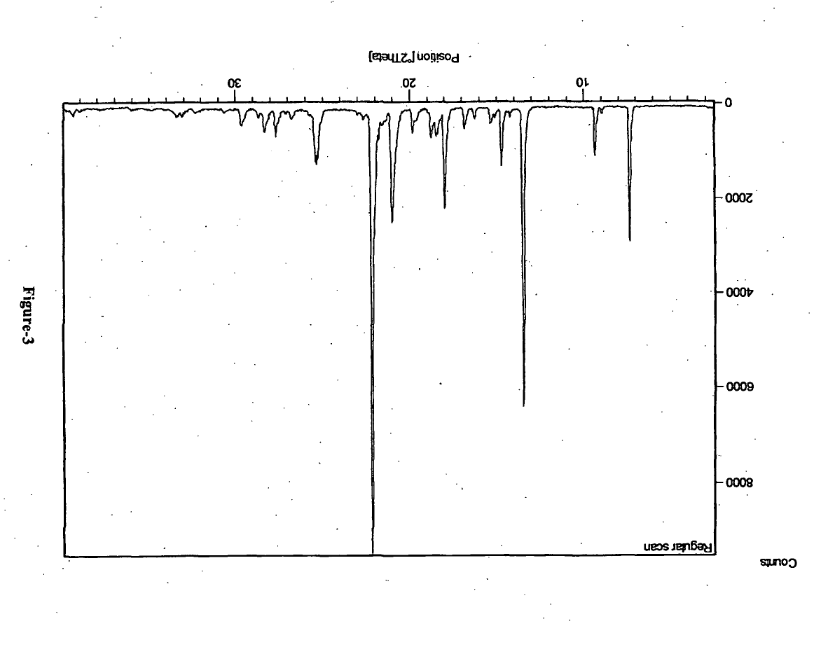

FIG. 3: PXRD graph of Form I obtained by following Example 9.

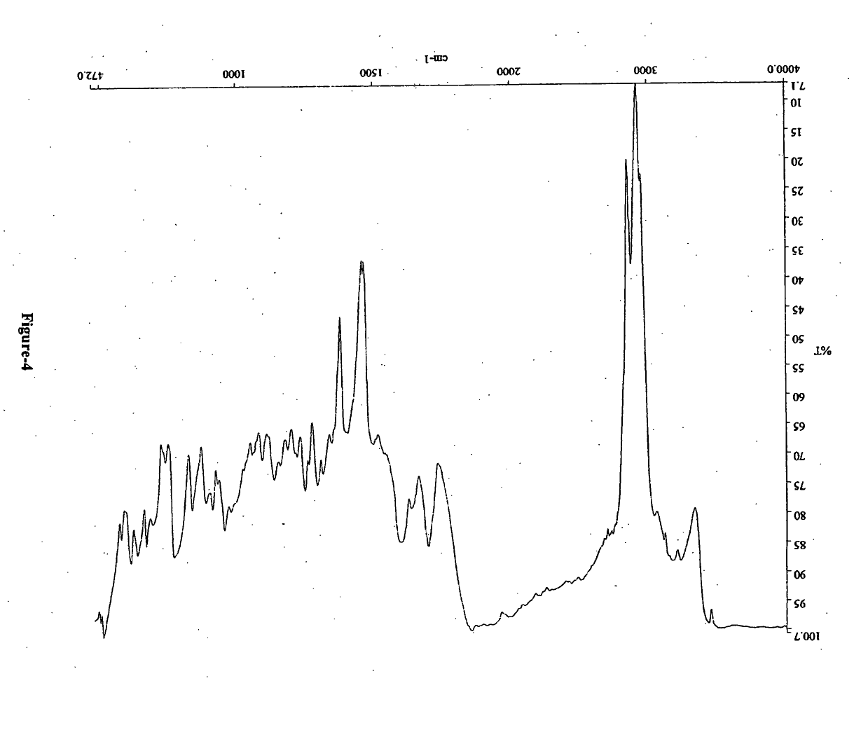

FIG. 4: FTIR (Nujol) Form I obtained by following Example 9.

### DETAILED DESCRIPTION OF THE INVENTION

The present invention provides a process for the preparation of Linezolid of formula (I) comprising a step of

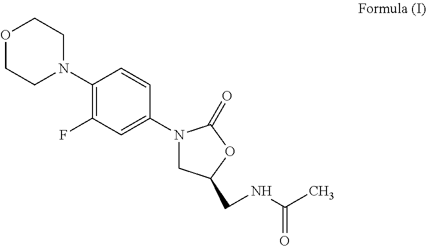 [SMILES: CC(=O)NC[C@H]1CN(c2ccc(N3CCOCC3)c(F)c2)C(=O)O1]

reacting compound of formula (IV), wherein R is Hydrogen or Nitrogen protecting group, with compound of formula (III) in presence of n-butyl lithium and n-butanol in a suitable solvent to obtain compound of formula (II)

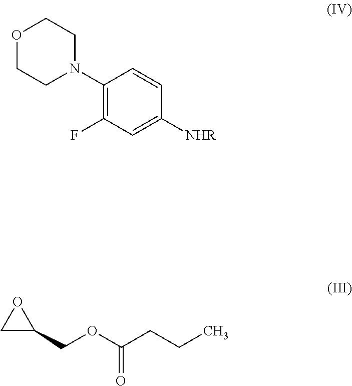

Another preferred embodiment of the present invention provides an improved process for the preparation of compound of formula (II) comprising of

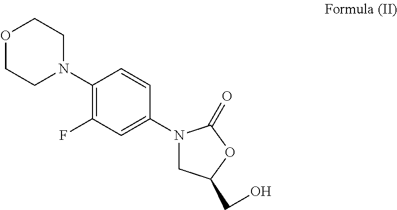 [SMILES: O=C1O[C@@H](CO)CN1c1ccc(N2CCOCC2)c(F)c1]

reacting compound of formula (IV)

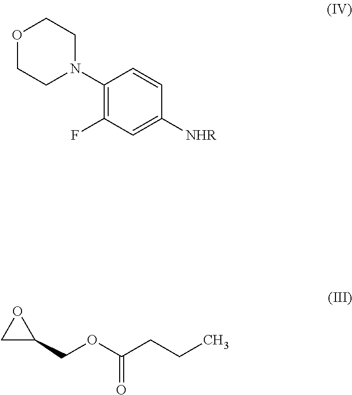

wherein R is Hydrogen or Nitrogen protecting group,

with compound of formula (III)

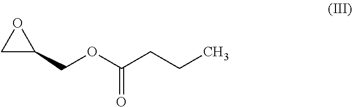

in presence of n-butyl lithium and n-butanol in a suitable solvent to obtain compound of formula (II)

The compound of formula (IV) can be prepared by any process disclosed in the prior art or methods known perse.

In one of the preferred embodiment compound of formula (IV) wherein R is Nitrogen protecting group preferably carbobenzoxy group is prepared by the process disclosed in Scheme-II. The compound of formula (IV) is converted to compound of formula (II) by reacting compound of formula (IV), with compound of formula (III) i.e. (R)-(−)-Glycidyl butyrate in the presence of n-butyl lithium and n-butanol in a suitable solvent.

The example of suitable solvent includes but is not limited to tetrahydrofuran. The reaction is carried out in the temperature range of −30° C. to 30° C. The reaction proceeds via formation of lithium salt of n-butanol.

After the completion of reaction the reaction mass is worked-up and the product obtained is used as such without further purification for the next step.

Compound of formula (II) is converted to Linezolid via formation of mesylate which is converted to azide by the methods knows perse. The azide is reduced and acylated to obtain Linezolid.

In another embodiment of the present invention, Linezolid of formula (I)

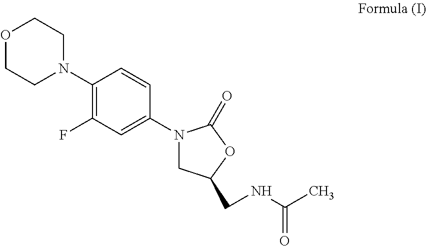 [SMILES: CC(=O)NC[C@H]1CN(c2ccc(N3CCOCC3)c(F)c2)C(=O)O1]

is prepared by process comprising acylating Linezolid amine of formula (Ia)

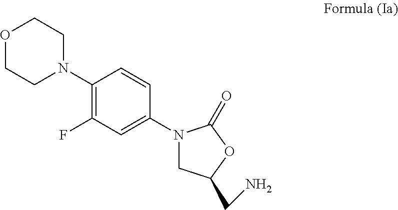 [SMILES: NC[C@H]1CN(c2ccc(N3CCOCC3)c(F)c2)C(=O)O1]

using acylating agent in the presence of ketonic solvent.

As used herein, acylating agent refers to acetic anhydride, acetyl chloride, acetic acid or any such reagent which is capable of introducing acetyl group.

As used herein, ketonic solvents refers to acetone, methyl iso-butyl ketone, methyl ethyl ketone, and the like or mixtures thereof.

The step of acylation is carried out at about 0° C. to about room temperature, preferably at about 0°-5° C.

Linezolid amine of formula (Ia) can be used directly or without isolation after the step of reduction from Linezolid azide of formula (IIb). The step of reduction of Linezolid azide of formula (IIb) is preferably carried out using palladium on carbon in presence of ethyl acetate as solvent.

In a preferred embodiment of the present invention, a one pot process is provided wherein Linezolid amine of formula (Ia) is not isolated from the reduction mixture, but the residue obtained after removal of catalyst and solvent used for reduction step, is converted to Linezolid of formula (I) by acylation using acylating agent in the presence of ketonic solvent.

Following comparison table indicates the content of (R)-enantiomer, bis-Linezolid impurity and purity of Linezolid when acylation is carried out in ethyl acetate and acetone:

TABLE 1

| | | | | |
|---|---|---|---|---|
| | | UPLC | Bis-Linezolid | |
| Sr. No. | Batch No. | Purity | impurity | (R)-enantiomer |

Ethyl acetate

| | | | | |
|---|---|---|---|---|
| 01 | Batch-1 | 99.57 | 0.39 | 0.08 |
| 02 | Batch-2 | 98.91 | 0.22 | 0.42 |
| 03 | Batch-3 | 98.95 | 0.46 | 0.43 |

Acetone

| | | | | |
|---|---|---|---|---|
| 01 | Batch-1 | 99.62 | 0.12 | 0.03 |
| 02 | Batch-2 | 99.70 | 0.07 | 0.02 |
| 03 | Batch-3 | 99.95 | ND | 0.05 |

The data clearly indicates significant reduction in bis-Linezolid impurity and (R)-enantiomer when acylation reaction is carried out in the presence of acetone as solvent.

Therefore, Linezolid prepared by the process of the present invention has content of (R)-enantiomer less than about 0.1%, preferably less than 0.5%. Further, Linezolid prepared by the process of the present invention has content of bis-Linezolid less than about 0.15%. Also, the purity of Linezolid prepared by the process of the present invention is greater than 99%, preferably greater than 99.5%.

Further, embodiment of the present invention provides a process for preparing Form I of Linezolid of formula (I) comprising steps of:

(a) acylating Linezolid amine of formula (Ia) using acylating agent in the presence of ketonic solvent.

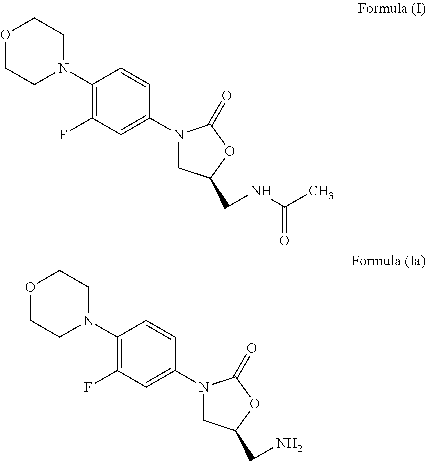 [SMILES: CC(=O)NC[C@H]1CN(c2ccc(N3CCOCC3)c(F)c2)C(=O)O1 ; NC[C@H]1CN(c2ccc(N3CCOCC3)c(F)c2)C(=O)O1]

(b) crystallizing Linezolid obtained in step (a) from suitable solvent.

In a preferred embodiment, crystallization of linezolid is preferably carried out in the presence of n-propanol or methyl isobutyl ketone.

In another preferred embodiment of the present invention, Linezolid azide of formula (IIb) is reduced using palladium on carbon in presence of ethyl acetate as solvent. After completion of the reaction the reaction mass is filtered and ethyl acetate is removed from the filtrate. In the same pot i.e. without isolating or further purifying the Linezolid amine of formula (Ia), acetone is added followed by acetic anhydride and triethyl amine at about 0-5° C. Then, the reaction mass is heated to reflux at about 65-75° C. followed by cooling at bout 0-5° C. to obtain a solid which is isolated by conventional methods like filtration, centrifugation and the like and dried. The solid thus obtained in dissolved in n-propanol and treated with activated charcoal and filtered.

The filtrate is concentrated and cooled to about 0-5° C. to obtain Linezolid Form I.

The synthetic reaction scheme of the present invention is as shown below.

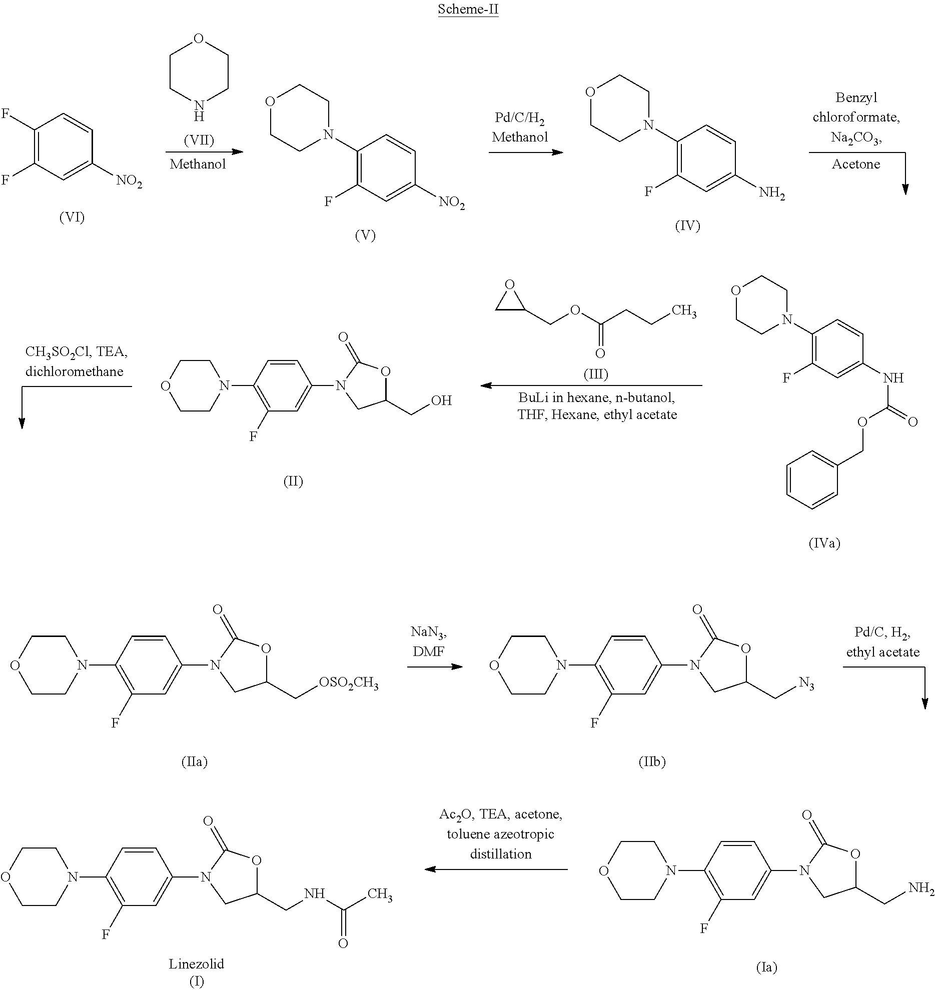

The advantages of process of present invention are:

1. It does not require temperature as low as −78° C., which is practically difficult to maintain during scale up process.

2. The product obtained does not require further purification by cumbersome process such as column chromatography which is difficult to perform at commercial scale.

The following examples illustrate the invention further. It should be understood however, that the invention is not confined to the specific limitations set forth in the individual example but rather to the scope of the appended claims.

### EXAMPLES

### Example 1

### Preparation of 3-Fluoro-4-morpholinyl nitrobenzene

To a solution of Methanol (90 ml) and 3,4-Difluoronitrobenzene (100 g) at 25-30° C. add Morpholine (115 g) drop wise at 25-30° C. in more than 1 hour under stirring. Stir the reaction mass at 25-30° C. for 1-2 hours. Then add slowly Water (400 ml) with stirring the reaction mass at 25-30° C. for 1 hour. Filter the solid & wash it with water. The solid is dried at 55-60° C. Yield: 1.408.; Percentage 99.0% w/w.

### Example 2

### Preparation of N-Carbobenzoxy-3-fluoro-4-morpholinylaniline

Take 3-Fluoro-4-morpholinyl nitrobenzene (100 g), Methanol (1000 ml) and 10% palladium on carbon catalyst (2.0 g 50% wet) in the autoclave at 20-30° C. for 3-4 hrs at 1-2 kg hydrogen pressure. Filter it and wash the hyflo bed by methanol (50 ml×2). Apply vacuum to remove traces of methanol & add Acetone (100 ml) to distill out completely below 70° C. Cool it & further add Acetone (400 ml) and sodium carbonate (46.9 g) to the residue. After cooling the mix at 0-5° C., 166 g of Benzyl chloroformate (50% solution in Toluene) was added slowly at 0-5° C. under stirring. Water (800 ml) & n-Hexane (100 ml) are added at 0-5° C. for 1 hour at constant stirring. The mixture was filtered & solid was washed with water (200 ml×2) and n-Hexane (100 ml). The solid is dried at 55-60° C. Yield: 1.43.; Percentage 97.9% w/w.

### Example 3

### Preparation of (R)-[N-3-(3-Fluoro-4-morpholinylphenyl)-2-oxo-5-oxazolidinyl]methanol

Take n-Butanol (51.5 g) and THF (100 ml) at 20-30° C. under Nitrogen atmosphere. After cooling the mix add slowly n-Butyl lithium (1.6M in hexane) (391.7 g) at 10 to 20° C. & maintain it for 45-60 minutes. Take THF (500 ml) and N-Carbobenzoxy-3-fluoro-4-morpholinylaniline (100 g) at 20-30° C. under Nitrogen atmosphere. Cool the mix at −15 to −5° C. under stirring. To this solution add slowly n-Butyl lithium solution & maintain for 45-60 minutes at −15 to −5° C., to this solution add slowly (R)-(−) Glycidyl butyrate (48.0 g) & maintain for 1 hour at −10 to −5° C. After completing addition raise the temperature to 8-13° C. and maintain for 1 hour & then take it to 13-15° C. and maintain for 4-5 hours. Organic layer was separated by water (800 ml) and Ethyl acetate (300 ml). Filter & wash the solid with mix of Ethyl acetate-n-Hexane & dried in air tray dryer at 55-60° C. Yield: 0.765.: Percentage 85% w/w.

### Example 4

### Preparation of (R)-[N-3-(3-Fluoro-4-morpholinylphenyl)-2-oxo-5-oxazolidinyl]methyl methane sulfonate

Triethyl amine (68.2 g), Methane sulfonyl chloride (48.3 g) are added to a flask containing Dichloromethane (1900 ml) and (R)-[N-3-(3-Fluoro-4-morpholinylphenyl)-2-oxo-5-oxazolidinyl]methanol (100 g) at 20-30° C. with constant stirring for 2-3 hours. After cooling & filtration wash the solid with Dichloromethane followed by water wash & dried in air tray dryer. Yield: 1.20.: Percentage 95% w/w.

### Example 5

### Synthesis of (R)-[N-3-(3-Fluoro-4-morpholinylphenyl)-2-oxo-5-oxazolidinyl]methyl azide

Reflux the mix of Dimethyl formamide (250 ml), (R)-[N-3-(3-Fluoro-4-morpholinyl phenyl)-2-oxo-5-oxazolidinyl]methyl methane sulfonate (100 g) and Sodium azide (24.3 g) at 60-65° C. & maintain it for 6-7 hours. Cool the mix & add water (450 ml) with constant stirring for one hour at 20-30° C. Filter it; wash the solid with mix of Dimethyl formamide-water (1:1) and with water & dried at 55-60° C. Yield: 0.82.: Percentage 95% w/w.

### Example 6

### Synthesis of Linezolid Crude

Ethyl acetate (3500 ml) and 10% palladium on carbon catalyst (6.0 g) are added in autoclave having (R)-[N-3-(3-Fluoro-4-morpholinylphenyl)-2-oxo-5-oxazolidinyl]methyl azide (100 g) at 20-30° C. Cool the reaction mass & maintain 2-3 kg hydrogen pressure at 15-20° C. for 6-7 hrs. Filter it & wash the hyflo bed by Ethyl acetate (100 ml×2). Then add the Triethyl amine (35.1 g) & Acetic anhydride (29.9 g) slowly at 25-30° C. under stirring. Cool the mix, filter it and wash the solid with chilled (0-5° C.) Ethyl acetate (100 ml) followed by water (100 ml×2). Finally product is dried at 55-60° C. Yield: 0.85.: Percentage 81% w/w.

### Example 7

### Synthesis of Linezolid Pure

Reflux the Acetone (1020 ml) and Linezolid crude (100 g) at 55-60° C. for the 30 minutes. Filter the hot turbid solution & wash it with hot (55-60° C.) acetone (50 ml). Cool the reaction mixture at −5 to 0° C. for 1 hour, wash the solid with chilled (−5 to 0° C.) acetone (50 ml). After drying the Linezolid semi pure (77 g) add n-Propanol (308 ml) reflux it at 95-100° C. for 30 min & filter it by hot solution through hyflo bed. Cool the mix to 0-5° C. for 1 hour and wash the solid with chilled (0-5° C.) n-Propanol (77 ml). Dry the material at 55-60° C. Yield: 0.73.: Percentage 73% w/w.

### Example 8

### Synthesis of Linezolid

Ethyl acetate (3500 ml) and 10% palladium on carbon catalyst (6.0 g) are added in autoclave having (R)-[N-3-(3-Fluoro-4-morpholinylphenyl)-2-oxo-5-oxazolidinyl]methyl azide (100 g) at 20-30° C. Cool the reaction mass & maintain 2-3 kg hydrogen pressure at 15-20° C. for 6-7 hrs. Filter it & wash the hyflo bed by Ethyl acetate. Distill out ethyl acetate at 75-90° C. and then cool the reaction mass to 0-5° C. Add acetone (1000 ml) & acetic anhydride (29.9 g) at 0-5° C. Further, add Triethyl amine (37.8 g) slowly at 0-5° C. under stirring. Maintain the reaction mass at 0-5° C. for 1-2 hrs. Heat the reaction mass to reflux at 65-75° C. for 1 hr. Again cool the reaction mass to 0-5° C. for 1 hr. Filter the solid wash it with acetone and water and dry it at 55-60° C. Yield: 0.80.: Percentage 80% w/w.

### Example 9

### Synthesis of Linezolid Form I

Reflux n-propanol (400 ml) and Linezolid (100 g) at 95-100° C. till all solid gets dissolved. Add activated charcoal (2.0 g) and heat for 30 mins. Filter thro hyflo bed. Heat the filtrate and concentrate the solution by partially removing n-propanol. Cool to 0-5° C. and filter the solid and dry it at 55-60° C. under vacuum. Yield: 0.9.: Percentage 90% w/w.

1. A method for the preparation of a Linezolid of formula (I), 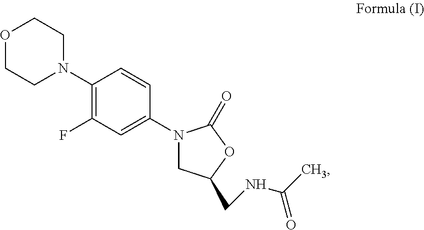 [SMILES: CC(=O)NC[C@H]1CN(c2ccc(N3CCOCC3)c(F)c2)C(=O)O1]
comprising the steps of:
reacting a compound of formula (IV), 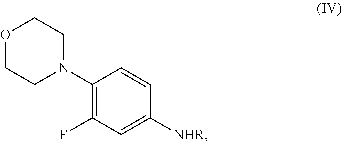
wherein R is Hydrogen or a Nitrogen protecting group, with a compound of formula (III), 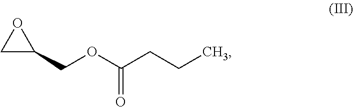
in the presence of n-butyl lithium and n-butanol in a suitable solvent at a temperature of −30° to 30° C. to obtain a compound of formula (II), 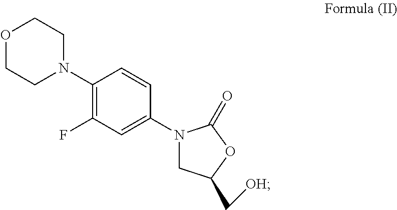 [SMILES: O=C1O[C@@H](CO)CN1c1ccc(N2CCOCC2)c(F)c1]
and converting the compound of formula (II) to Linezolid of formula (I).

2. A method for the preparation of a compound of formula (II) 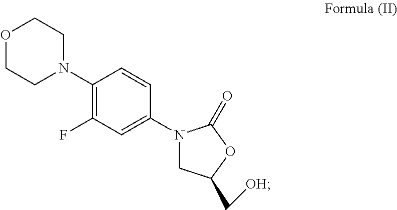 [SMILES: O=C1O[C@@H](CO)CN1c1ccc(N2CCOCC2)c(F)c1]
comprising the step of:
reacting a compound of formula (IV), 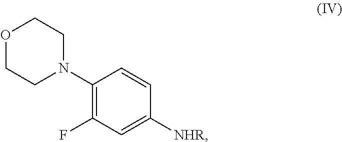
wherein R is Hydrogen or a Nitrogen protecting group, with a compound of formula (III), 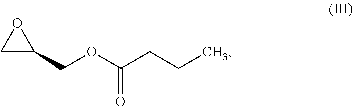
in the presence of n-butyl lithium and n-butanol in a suitable solvent at a temperature of −30° to 30° C. to obtain the compound of formula (II).

3. The method of claim 1 or 2, wherein said suitable solvent is tetrahydrofuran.

4. (canceled)

5. A method for the preparation of a Linezolid of formula (I) 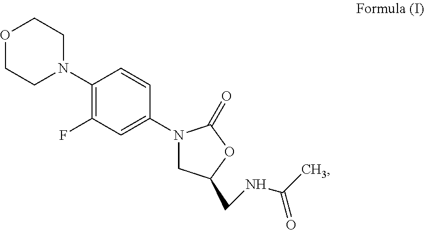 [SMILES: CC(=O)NC[C@H]1CN(c2ccc(N3CCOCC3)c(F)c2)C(=O)O1]
comprising the step of:
acylating a Linezolid amine of formula (Ia), 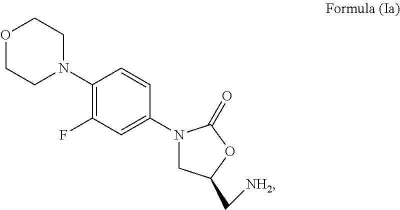 [SMILES: NC[C@H]1CN(c2ccc(N3CCOCC3)c(F)c2)C(=O)O1]
acylating-Linezolid amine of formula (Ia) using an acylating agent in the presence of a ketonic solvent.

6. A method for the preparation of a Linezolid of formula (I), 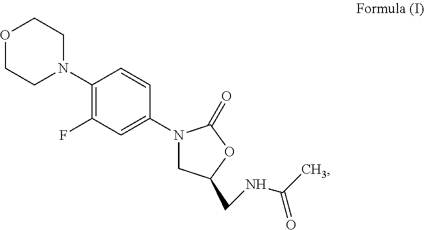 [SMILES: CC(=O)NC[C@H]1CN(c2ccc(N3CCOCC3)c(F)c2)C(=O)O1]
comprising the steps of:
(a) acylating a Linezolid amine of formula (Ia), 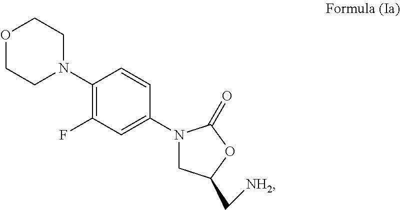 [SMILES: NC[C@H]1CN(c2ccc(N3CCOCC3)c(F)c2)C(=O)O1]
using an acylating agent in the presence of a ketonic solvent, and
(b) crystallizing the Linezolid obtained in step (a) from a suitable solvent, wherein the Linezolid of formula (I) is Form I characterized by the XRD of FIG. 1.

7. The method of claim 5 or 6, wherein said acylating agent is acetic anhydride or acetyl chloride.

8. The method of claim 5 or 6, wherein said ketonic solvent is acetone, methyl iso-butyl ketone, methyl ethyl ketone, or a mixture thereof.

9. The method of claim 6, wherein said crystallization is carried out in n-propanol or methyl iso-butyl ketone.

10. A Linezolid of formula (I) obtained by the method of claim 5 or 6, having content of (R)-enantiomer less than about 0.1% and content of bis-Linezolid less than about 0.15%.

11. A Linezolid of formula (I) obtained by the method of claim 5 or 6, having purity greater than 99%.
